(19) United States
(12) Patent Application Publication
(10) Pub. No.: US 2006/0036327 A1
(43) Pub. Date: Feb. 16, 2006

(54) Prosthetic intervertebral disc implant

(76) Inventor: Albert Enayati, Paramus, NJ (US)

Correspondence Address: Michael G. Petit, P.O. Box 91929, Santa Barbara, CA, 93190-1929, US

(21) Appl. No.: 11/028,800

(22) Filed: Jan. 3, 2005

Related U.S. Application Data
(63) Continuation-in-part of application No. 10/916,989, filed on Aug. 11, 2004, now abandoned.

(51) Int. Cl. A61F 2/44

(52) U.S. Cl. 623/17.15; 623/17.14

(57) ABSTRACT

A prosthetic disc for the replacement of a defective intervertebral disc comprising a lower component member and a upper component member, each component member having three distinct complementary bearing surfaces which allow anterior-posterior (bending forward/backward), medial-lateral (bending side-to-side), and twisting motions approximating the natural movement of a spinal joint. The lower component member has three adjacent bearing surfaces which are each longitudinally and medial-laterally convex. The upper component member has three substantially complementary longitudinally and medial-laterally concave bearing surfaces. When the prosthesis is disposed between adjacent vertebrae, it provides medial-lateral support while allowing anterior-posterior, medial-lateral and twisting motions approximating the natural movement of the spine.

### BACKGROUND OF THE INVENTION

1. Field of the Invention

The present invention relates to a prosthetic intervertebral disc for a human spine.

2. Prior Art

Human spines are formed from a stack of vertebrae, which are separated and cushioned from each other by intervertebral discs. The discs consist of a fibrous outer envelope containing a gel-like fluid. The discs are subject to large compressive forces, which may vary from about 175 pounds when a person is at rest to as high as about 500 pounds. For example, a person who lifts a 15 pound weight one foot in front of such person, using a bending movement, can generate nearly 500 pounds of force on his or her spine. Because of the high compressive forces they must withstand, spinal discs commonly rupture, particularly as they deteriorate with age.

Various attempts have been made to deal with the problem of a ruptured disc. One standard procedure is to remove the disc and fuse the vertebrae which were formerly separated by the disc. A difficulty with this surgical approach is that relative motion between the two vertebrae is no longer possible, causing both stiffness in the spine and referred difficulties in areas above and below the fused vertebrae.

Other attempts to deal with the problem have involved removing the ruptured disc and replacing it with an artificial resilient pad made, for example, of high density polyethylene or of a silicone material. Such pads tend to wear out rapidly, since a spinal disc typically undergoes goes between one and five million cycles of compression and extension per year. In addition pads provide no compliance or natural restoring force, and they do not provide the constraints to movement, which are provided by a natural disc. Another proposed solution is to use ball bearings in place of discs. However no satisfactory method has been developed for retaining the ball bearings in place.

There have been numerous types of implants designed to replace damaged spinal disc segments of the human body in which an artificial intervertebral disc is used to replace a deformed, injured or diseased natural intervertebral disc. Such an artificial intervertebral disc is generally composed of a rigid solid body that causes the vertebrae adjacent to the implanted artificial disc to be limited in its ability to move relative to each other. Some of these solid body artificial intervertebral discs are disclosed in U.S. Pat. Nos. 4,349,921; 4,553,273; and 4,714,469. Other artificial discs are provided with a spring which permits the vertebrae adjacent to the implanted artificial disc to have a limited range of motion. These are described in U.S. Pat. Nos. 4,309,777 and 4,759,769.

It has also been proposed that soft compliant polymers be used to assure separation of damaged disc segments or as bearing surfaces. However, polymers can degrade with time in the human body and may be susceptible to abrasion damage by the bone surface. Further, polymeric disc replacements are susceptible to creep and gradual changes in dimensional characteristics. Nevertheless, polymers are favored due to their low stiffness and relative improvement in shock absorbing characteristics. Artificial intervertebral discs having a main body formed of an elastic polymer are disclosed in U.S. Pat. Nos. 3,867,729; 4,863,477 and 4,911,71. U.S. Pat. No. 5,071,437 discloses an artificial intervertebral disc composed of an elastomeric core sandwiched between two metal plates. This disc is further provided with a plurality of spikes for stabilizing the vertebrae adjacent to the implanted disc. However, the biocompatability of this disc is uncertain because it contains a polymer and a curing agent used to hold the metal plates and the elastomeric core together.

Ball and socket type artificial spinal discs have been proposed, but without polymer components they have less shock absorbing capabilities. Further, there is no clinical proof that shock absorption is necessary, as adjacent discs can compensate for a reduced level of shock absorption in one or two discs along the entire spinal column.

Non-polymeric disc components include ceramic and metal materials and designs have included hinged, sliding and ball and socket-type constructions. The hinged design frequently is constraining, and the patient cannot effectively move in all directions. Sliding disc surfaces generally do not have the ability to accommodate bending or twisting motion, but do assist with natural translational motion within the disc space. However, due to the small space between adjacent vertebrae, the contact stress with only this type of single (translational) motion capability between the sliding surfaces can lead to excessive wear and eventual disfunction of the artificial intervertebral disc.

Other designs using either ceramic or metal materials also restrict motion relative to the adjacent vertebrae. Some artisans believe that because the natural healthy disc experiences a limit of about 11 degrees of motion in the anterior-posterior plane (bending forward/backward) and a limit of about 3-5 degrees of motion in the medial-lateral plane (bending side-to-side), the artificial disc replacement must also have this limitation, which causes other adjacent discs to take up the strain. Further, some skilled in the art argue that there must be a restriction to rotation (1-2 degrees) within the artificial disc for the same reasons. For example, U.S. Pat. No. 4,759,769 discloses a hinged/spring design with a limitation of 20 degrees in flexion/extension that allows a small amount of side-to-side rocking. Rotation is restricted with this device. U.S. Pat. No. 5,071,437 describes an implant device that limits flexion/extension and lateral bending to physiologic angles and allows for only 2-3 degrees of rotation. This device also allows for 1-2 mm of translational motion.

U.S. Pat. No. 4,759,766 describes a device composed of two cobalt/chrome end plates with a domed polyethylene central core which allows 10 degrees of flexion/extension, a small amount of lateral bending, and is rotationally restricted. The addition of these anatomical restrictions to disc replacement designs limit the type and effectiveness of the design and the materials that may be employed. For example, if the disc prosthesis exceeds its 11 degree bending restriction, then, as mentioned above, the non-bearing surfaces will contact each other and inappropriately displace the prosthesis or cause adverse wear.

The present inventor posits that restrictions in disc prosthesis design may not only create a performance liability, but are also not needed. The surrounding ligaments, muscles, and other tissues provide a built-in restriction to anterior/posterior, medial-lateral, and twisting motions of the artificial intervertebral disc, at least to a point in which adjacent discs are not significantly affected.

For the reasons mentioned above, the present invention provides a prosthetic disc comprising an upper member and a lower member having three distinct adjacent concave articular surfaces having low wear bearing metal surfaces, precision machined to minimize contact stress and anchored to the adjacent vertebrae with press fit or cemented methods known in the art of joint replacement. To accommodate extreme motion, the three distinct adjacent articulating surfaces are designed to minimize stress and wear. Additionally, the bearing portions of the disc of the artificial intervertebral disc can be attached to a different material for anchoring the disc portions to the adjacent bone.

### SUMMARY

The present invention is directed to a prosthetic intervertebral disc and a method for using the prosthetic disc that substantially obviates one or more of the limitations of the related art. To achieve these and other advantages and in accordance with the purpose of the invention, as embodied and broadly described herein, the invention includes an artificial intervertebral disc, which will offer improved performance and a longer lifetime in a patient.

It is an object of the present invention to provide a prosthetic implantable disc for a human spine that provides a greater range of motion to the spine than provided by prior art prosthetic discs.

The prosthetic disc in accordance with the present invention is adapted to replace a natural disc and to be implanted between two adjacent vertebrae. The prosthetic disc comprises a lower component member and an upper component member. The lower component member has three adjacent bearing surfaces which are each longitudinally and medial-laterally convex. The upper component member is provided with three substantially complementary longitudinally and medial-laterally concave bearing surfaces that provide medial-lateral support while allowing anterior-posterior, medial-lateral and twisting motions approximating the movement of the natural spine. The prosthetic disc allows a anterior-posterior and medial-lateral motion angle of about 10 to 40 degrees and a twisting angle of between 1 and 5 degrees. The width of the central surface of the upper component member is slightly wider than the corresponding surface for the lower component member to allow approximately 1 to 5 degrees of rotation. The upper component member and the lower component member have means for attachment to the upper and lower vertebrae respectively, producing a stable joint with a range of motion approximating that of the natural spine joint.

Other objects, advantages and salient features of the present invention will become apparent from the following detailed description, which, taken in conjunction with the annexed drawings, discloses preferred embodiments of the present invention. The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is an exploded, perspective view illustrating the relationship of the disc prosthesis relative to the upper and lower vertebrae;

FIG. 2 is a perspective view of the disc prosthesis;

FIG. 3 is a top plan view of the upper member of the prosthesis;

FIG. 4 is a bottom plan view of the lower member of the prosthesis;

FIG. 5 is a sectional view of the prosthesis taken along lines 5-5 of FIG. 6;

FIG. 6 is a sectional view of the prosthesis taken along lines 6-6 of FIG. 5;

FIG. 7 is a lateral view, partly in section of a spine showing the relationship of the upper and lower members of the prosthesis; and

FIG. 8 is a view, partly in section from the posterior of the spine as seen from the left side of the spine of FIG. 7.

FIG. 9 is a top perspective view of an intervertebral disc prosthesis implant in accordance with the present invention wherein housings are disposed on a bone contacting surface operable for containing bone graft material and permitting host bone ingrowth into the implanted prosthesis.

FIG. 10 is an exploded plan view of the intervertebral disc prosthesis shown in FIG. 9.

### DESCRIPTION OF THE PREFERRED EMBODIMENTS

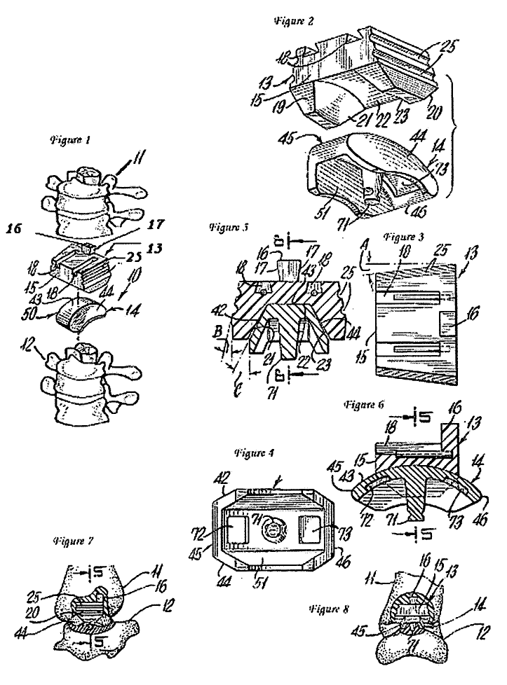

Referring to the drawings in further detail as shown in FIGS. 1-8, there is seen a disc joint prosthesis which is constructed in accordance with the principles of this invention and designated generally by reference number 10. In FIG. 1 the prosthesis 10 is shown in relation to its connection to the upper vertebra 11 and the lower vertebra 12, respectively. The prosthetic disc 10 is formed by an upper member 13 inserted into the upper vertebra 11 and a lower member 14 inserted into the lower vertebra 12. The facing surfaces of the upper vertebra 11 and the lower vertebra 12 are prepared with a minimum of bone removal by cutting and/or reaming the facing surfaces thereof so that the prosthetic members 13 and 14 can be affixed to the corresponding bones.

With continued reference to FIG. 1 and/or FIGS. 2-5, the upper member 13 may be fabricated from biologically compatible material such as high density polyethylene high or ultra-high molecular weight polyethylene or a hybrid material. The upper member 13 consists of a generally trapezoidal-shaped body 15. Integrally joined to and extending upwardly from approximately the center of the forward edge of the trapezoidal-shaped body 15 is a lug 16 having tapered sides 17 converging toward trapezoidal-shaped body 15. The upper surface of the trapezoidal-shaped body 15 contains dovetailed tracks 18 oriented in the anterior/posterior direction. The tracks 18 and lug 16 are designed for cement fixation. The lower surface of the trapezoidal-shaped body 15 and sidewalls 19 and 20 (FIG. 2) define three distinct concave, both longitudinally and laterally, bearing surfaces 21, 22 and 23. The external surfaces 19 and 20 of the upper member 13 are tapered. The width of the superior external surface decreases posteriorly the angle A (FIG. 3) being 1 degree to 40 degrees, and preferably about 5 degrees, and conforms to the generally trapezoidal cross-section of the upper vertebra and provides a good fit in bone. The width of the upper member increases inferiorly which corresponds to angle B (FIG. 5), preferably 10 degrees. Grooves 25 are located in the external surface of sidewalls to allow bone cement fixation. The grooves 25 are parallel and oriented in the anterior/posterior direction.

The contour of the three concave articulating surfaces 21, 22 and 23 (FIG. 2) of the upper component member 13 matingly match the contour of the three articulating surfaces on the lower member quite closely as will be discussed below. The width of the central surface of the upper component member is slightly wider (about 0.015 inches) than the corresponding surface in the lower component. This difference permits approximately 1 to 5 degrees of rotation between the upper member 13 and the lower member 14. The lateral surfaces 21 and 23 of the upper component cover approximately 70% of the corresponding surfaces 42 and 44 of the lower component.

The lower member 14 may be fabricated from a biologically compatible metal or metal alloy consisting of cobalt, chromium and molybdenum or a hybrid material and includes an upper portion 50 and lower attachment portion 51 wherein the upper portion 50 is provided with bearing surfaces 42, 43 and 44 which are convex and complementary in contour to bearing surfaces 21, 22 and 23 of the upper member. The bearing surfaces 42, 43 and 44 are both longitudinally and laterally convex. The length of the central or upper bearing surface 43 of the lower member, when viewed from its medial side as seen in FIG. 8, is generally longitudinally and laterally convex and the lateral bearing surfaces 42 and 44 are also longitudinally and laterally convex and are inclined at and angle C (FIG. 5) from 5-70 degrees and preferably from 25-70 degrees, and most preferably, 30 degrees from the vertical.

The anterior and posterior edges 45 and 46 of the lower member 14 are blunt to prevent scraping of the bone or soft tissue anterior or posterior to the metal prosthesis. Located on the underside of the central surface of the lower member is a short post 71 (FIGS. 5 and 6) for cement fixation in bone. The post 71 is located centrally and, in addition, two rectangular depressions 72 and 73 are located anteriorly and posteriorly to this post. These depressions allow bone cement to “key” in the prosthesis 10. All three bearing surfaces are load-bearing and the lateral surfaces limit lateral and medial movement of the lower component thereby providing medial-lateral stability.

In use, the upper component articulates on the lower component and provides for anterior-posterior, medial-lateral motions. The anterior/posterior length of the upper component is shorter than the lower component. This allows the upper component to ride anteriorly and posteriorly over the lower component.

The range of motion provided by this prosthesis is about 40 degrees. A limited amount of rotation is permitted by the clearance created between the central articulating surfaces 22 and 42 of the upper and lower components respectively.

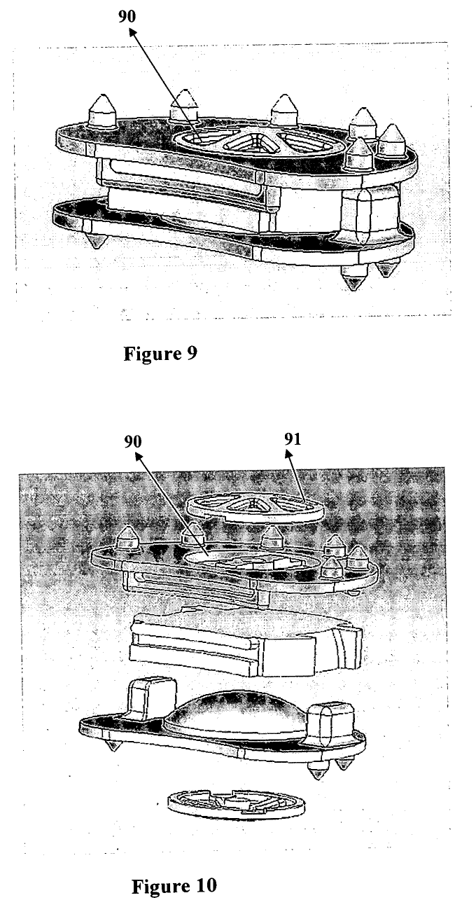

FIG. 9 is a top perspective view of an intervertebral disc prosthesis implant in accordance with the present invention wherein housings are disposed on a bone contacting surface operable for containing bone graft material and permitting host bone ingrowth into the implanted prosthesis. FIG. 10 is an exploded plan view of the intervertebral disc prosthesis shown in FIG. 9.

In practice, the surgical procedure preferably involves cutting out a section of the anterior upper vertebra and cutting a notch in the central part of the upper vertebr 11 to accommodate lug 16. Through the use of a template and drill, a bore to accommodate post 71 is provided in the lower vertebra 12 and the lower vertebra is contoured to accommodate the lower component 12. Cement, e.g., methyl methacrylate cement, is then applied to the site and the lug and post entered into their corresponding bone notches and bore.

While particular embodiments of the present invention have been illustrated and described, it would be obvious to those skilled in the art that various other changes and modifications can be made without departing from the spirit and scope of the invention. For example, either the upper or the lower member (or both) can be made, either in part or completely, of a hybrid material. It is therefore intended to cover in the appended claims all such changes and modifications that are within the scope of this invention.

What I claim is:

1. An prosthetic disc for surgical placement between upper and lower adjacent vertebrae of a patient's spine, comprising: (a) a lower component member having three adjacent longitudinally and medial-laterally convex load-bearing surfaces; (b) means for attaching said lower component member to the lower vertebra bone; (c) an upper component member having three longitudinally and medial-laterally concave load-bearing surfaces that are substantially complementary in contour to said convex load-bearing surfaces, and (d) means for attaching said upper component member to the upper vertebrae bone; wherein said convex surfaces and said concave surfaces are in mutual articulating bearing engagement and provide medial-lateral support to the upper and lower adjacent vertebrae.

2. The prosthesis according to claim 1 wherein said respective concave and convex load-bearing contoured surfaces are configured so as to allow anterior-posterior, medial-lateral and twisting motions approximating the movement of the natural spine joint between the upper and lower components relative to a patient's normal spinal axis when in a standing position with the spine substantially straightened, said convex and concave contoured surfaces allowing for a anterior-posterior and medial-lateral motion angle of about between 10 to 40 degrees and twisting motion angle of about between 1 and 5 degrees.

3. The prosthesis according to claim 1 wherein said concave load-bearing surface has a first central surface portion and said convex load-bearing surface has a second central surface portion and wherein the difference in width between the second central load-bearing surface of the lower component and the first central load-bearing surface of the upper component is sufficient to allow a twisting motion angle of about between 1 to 5 degrees of the lower component relative to the orientation of the upper component.

4. A prosthesis according to claim 2 wherein said convex load-bearing surfaces of the lower component member and said concave load-bearing surface of the upper component member each have medial-lateral surfaces adjacent said first and second central surface portions.

5. A prosthesis according to claim 1 wherein the upper member is fabricated from biologically compatible material selected from the group consisting of metals and elastomers or combinations thereof.

6. A prosthesis according to claim 1 wherein the lower member is fabricated from biologically compatible metal or metal alloy selected from the group consisting of cobalt, chromium and molybdenum.

7. A prosthesis according to claim 1 wherein the lower and upper member are fabricated from the same biologically compatible material or alloy selected from the group consisting of cobalt, chromium and molybdenum, or a biocompatible ceramic material.

8. A prosthesis according to claim 1, wherein the upper and lower component include anchoring means for attachment to the respective upper and lower vertebrae.

9. A prosthesis according to claim 8, wherein the anchoring means is selected from the group consisting of pegs, posts, rivet, slot and screws.

10. A prosthesis according to claim 8, wherein the anchoring means includes a surface selected from the group consisting of a porous-coated and a macro-textured surface or biological or synthetic coating to facilitate bone ingrowth.

11. A prosthesis according to claim 8 (FIG. 9) wherein said anchoring surface includes a housing 90 for containing bone graft material to permit ingrowth of host bone into said bone graft material thereby promoting fusion of said prosthesis to the adjacent vertebrae.

12. A prosthesis according to claim 8, wherein housing 90 has a cap 91 (FIG. 10.) The cap is made from selected group of materials consisting of a bioabsorbable material, molded polymer, ceramic, a pseudoelastic shape memory alloy, titanium, stainless steel or a cobalt chrome alloy. The bioabsorbable polymer base material selected from the group consisting of Poly-L-lactide and a co polymer of two or more poly-lactides such as 70:30 poly (L-lactide-co-D, L-lactide), PLA: PGA and a co polymer of two or more poly-lactides and Poly glycolides.
